(19) United States
(12) Patent Application Publication
(10) Pub. No.: US 2025/0160661 A1
(43) Pub. Date: May 22, 2025

(54) SPHYGMOMANOMETER

(71) Applicant: OMRON HEALTHCARE Co., Ltd., Kyoto (JP)

(72) Inventor: Yukiya SAWANOI, Kyoto (JP)

(21) Appl. No.: 19/029,227

(22) Filed: Jan. 17, 2025

Related U.S. Application Data
(63) Continuation of application No. PCT/JP2023/018717, filed on May 19, 2023.

(30) Foreign Application Priority Data
Sep. 9, 2022 (JP) 2022-143551

(51) Int. Cl. A61B 5/021; A61B 5/00; A61B 5/022

(52) U.S. Cl. CPC A61B 5/02116; A61B 5/02141; A61B 5/02225; A61B 5/7264; A61B 5/7282; A61B 2560/0462; A61B 2562/0247

(57) ABSTRACT

A blood pressure monitor including an information acquisition unit that acquires, for an individual blood pressure measurement, pulse wave feature information representing a feature amount of a pulse wave from a pulse wave signal superimposed on a cuff pressure signal detected in the blood pressure measurement, a storing unit that stores pulse wave feature information acquired for the individual blood pressure measurement, a pulse number determination unit that determines the number of pulse waves having a feature amount represented by pulse wave feature information for the individual blood pressure measurement, and an atrial fibrillation determination unit that determines atrial fibrillation in the pulse wave based on the pulse wave feature information in the storing unit when the total number of pulse waves for the individual blood pressure measurement reaches a predetermined number.

### CROSS-REFERENCE TO RELATED APPLICATIONS

This application is a continuation under 35 U.S.C. § 120 of International Application No. PCT/JP2023/018717, filed May 19, 2023, which claims priority to Japanese Application No. 2022-143551, filed Sep. 9, 2022, under 35 U.S.C. § 119(a). Each of the above-referenced patent applications are incorporated by reference in their entireties.

### TECHNICAL FIELD

The present disclosure relates to a blood pressure monitor, and more particularly, to a blood pressure monitor having a function of determining atrial fibrillation.

### BACKGROUND

Early detection of atrial fibrillation, which is a cause of heart disease, has been desired. Conventionally, a technique for estimating atrial fibrillation from pulse wave information acquired by an electronic blood pressure monitor for home use has been proposed. Specifically, in one measurement opportunity using the electronic blood pressure monitor, blood pressure measurement is continuously performed, for example, a plurality of times, a pulse wave interval which is an interval between pulse wave signals acquired in each blood pressure measurement is acquired, and atrial fibrillation is detected based on the pulse wave interval.

For example, US 2016/0228017 A (Patent Literature 1) discloses a blood pressure measurement device that can indicate the presence or absence of atrial fibrillation.

### SUMMARY

In the device disclosed in Patent Literature 1, in order to determine the presence or absence of atrial fibrillation, three consecutive blood pressure measurements are required in a single measurement occasion. When the blood pressure is continuously measured three times at every measurement opportunity in this manner, the time required for the measurement is increased, and the measurement site is pressed by the cuff to give a feeling of restraint to the user, which imposes a burden on the user.

An object of the present disclosure is to provide a blood pressure monitor that enables both blood pressure measurement and atrial fibrillation determination while reducing a burden on a user.

A blood pressure monitor according to the present disclosure includes: a cuff pressure adjustment unit configured to increase or decrease a cuff pressure indicating an internal pressure of a cuff attached to a measurement site of a user; a cuff pressure detector configured to detect a cuff pressure signal indicating the cuff pressure; a blood pressure measurement unit configured to measure a blood pressure of the user based on a pulse wave signal superimposed on a cuff pressure signal detected in a process of increasing or decreasing the cuff pressure; an information acquisition unit configured to acquire, for an individual blood pressure measurement, pulse wave feature information representing a feature amount of a pulse wave from the pulse wave signal superimposed on the cuff pressure signal detected in the blood pressure measurement; a storing unit configured to store pulse wave feature information acquired for the individual blood pressure measurement; a pulse number determination unit configured to determine the number of pulse waves having a feature amount represented by pulse wave feature information for the individual blood pressure measurement; and an atrial fibrillation determination unit configured to determine atrial fibrillation in a pulse wave based on the pulse wave feature information in the storing unit when the total number of the pulse waves for the individual blood pressure measurement reaches a predetermined number.

According to the above disclosure, atrial fibrillation in the pulse wave is determined based on the pulse wave feature information stored in the storing unit when the total number of pulse waves for an individual blood pressure measurement reaches a predetermined number. Accordingly, it is not necessary to perform the blood pressure measurement a plurality of times for the atrial fibrillation determination in a single blood pressure measurement opportunity. As a result, in the blood pressure measurement, it is possible to realize both the blood pressure measurement and the atrial fibrillation determination while reducing a burden on the user such as the measurement site of the user being repeatedly pressed a plurality of times or the blood pressure measurement time being increased.

The pulse number determination unit in the blood pressure monitor described above is configured to determine the number of pulse waves for the individual blood pressure measurement by excluding, from the pulse wave feature information for the individual blood pressure measurement, pulse wave feature information corresponding to a blood pressure measurement in which an elapsed time from when the blood pressure measurement is performed is equal to or greater than a threshold value.

According to the blood pressure monitor described above, old past pulse wave feature information can be excluded from the pulse wave feature information used for determining atrial fibrillation.

In the blood pressure monitor described above, the feature amount of the pulse wave represented by the pulse wave feature information includes an interval between pulse waves represented by the pulse wave signal superimposed on the cuff pressure signal detected in the blood pressure measurement.

According to the blood pressure monitor described above, the interval between pulse waves can be included in the feature amount of the pulse wave used for the atrial fibrillation determination.

In the blood pressure monitor described above, the interval between pulse waves includes a time interval between maximum values of amplitudes in adjacent pulse waves.

According to the blood pressure monitor described above, the time interval between maximum values of the amplitudes can be included as the interval between pulse waves.

In the blood pressure monitor described above, the pulse number determination unit configured to determine the number of pulse waves for the individual blood pressure measurement by excluding pulse wave feature information corresponding to a pulse wave in which an amplitude of a pulse wave having a feature amount represented by the pulse wave feature information for the individual blood pressure measurement is equal to or less than a threshold value.

According to the blood pressure monitor described above, it is possible to exclude pulse wave feature information corresponding to a pulse wave whose amplitude is equal to or less than a threshold value from pulse wave feature information used for determining atrial fibrillation.

In the blood pressure monitor described above, the pulse number determination unit is further configured to determine whether or not the total number of pulse waves corresponding to the pulse wave feature information for the individual blood pressure measurement in the storing unit reaches the predetermined number.

According to the blood pressure monitor described above, when the pulse number determination unit determines that the total number of pulse waves corresponding to the pulse wave feature information for the individual blood pressure measurement in the storing unit has reached the predetermined number, it is possible to determine atrial fibrillation in the pulse wave based on the pulse wave feature information stored in the storing unit.

According to the present disclosure, it is possible to perform both blood pressure measurement and atrial fibrillation determination while reducing a burden on a user.

### BRIEF DESCRIPTION OF THE DRAWINGS

FIG. 1 is a diagram illustrating an example of a network configuration to which a blood pressure measurement system according to the present embodiment is applied;

FIG. 2 is a block diagram illustrating an example of a hardware configuration of a blood pressure monitor 20 according to the present embodiment;

FIG. 3 is a diagram illustrating an example of a functional configuration of the blood pressure monitor 20 according to the present embodiment;

FIG. 4 is a flowchart illustrating an example of processing related to measurement by the blood pressure monitor 20 according to the present embodiment; FIG. 5 is a flowchart illustrating an example of the blood pressure measurement processing of FIG. 4;

FIG. 6 is a diagram illustrating a display example of information on a display 31 according to the present embodiment;

FIG. 7 is a diagram illustrating a display example of information on the display 31 according to the present embodiment; and

FIG. 8 is a diagram illustrating an example of a plurality of pulse waves used for an AF determination according to the present embodiment.

### DETAILED DESCRIPTION

Embodiments of the present invention will be described below with reference to the drawings. In the following description, like components are given like numerals. Names and functions thereof are also the same. Thus, the detailed description of such components is not repeated.

### Application Example

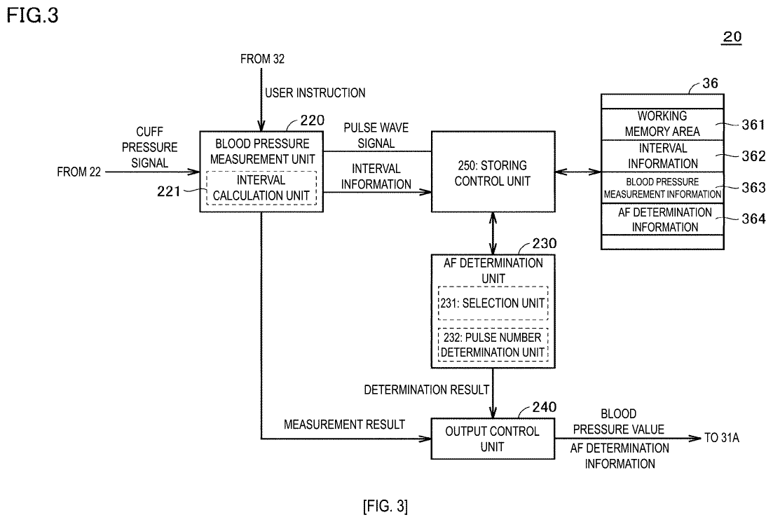

An application example of the blood pressure monitor according to this embodiment will be described with reference to FIG. 3. FIG. 3 is a diagram illustrating an example of the functional configuration of the blood pressure monitor 20 according to this embodiment. Hereinafter, atrial fibrillation will be referred to as AF.

The blood pressure monitor 20 according to the present embodiment is configured to measure a blood pressure by increasing or decreasing a cuff pressure indicating an internal pressure of a cuff (air bag) worn around a measurement site of a user, for example, an arm. The measurement site is not limited to the upper arm.

Referring to FIG. 3, the blood pressure monitor 20 includes, as main functional components, a blood pressure measurement unit 220, an AF determination unit 230, an output control unit 240 that controls output of information, and a storing control unit 250 that controls reading and writing of information from and to a storage unit 36. These units are realized by, for example, the processor 30 of the blood pressure monitor 20 reading a program stored in a storage such as a hard disk drive (HDD) 35 to be described later, expanding the read program in a memory 33 to be described later, and executing the program. Some or all of these units may be configured to be realized by hardware circuits including an ASIC (Application Specific Integrated Circuit) or an FPGA (Field Programmable Gate Array).

The storage unit 36 includes the HDD 35 to be described later or the memory 33 to be described later. The storage unit 36 includes a working memory area 361 mainly constituted by a volatile storage medium and an information memory area. In the memory area for this information, information including interval information 362, blood pressure measurement information 363 indicating information on the measured blood pressure, and AF determination information 364 indicating the determination result of AF is stored. The interval information 362 indicates an interval between pulse waves, which is an example of a feature amount of pulse waves detected in time series from a measurement site in a blood pressure measurement.

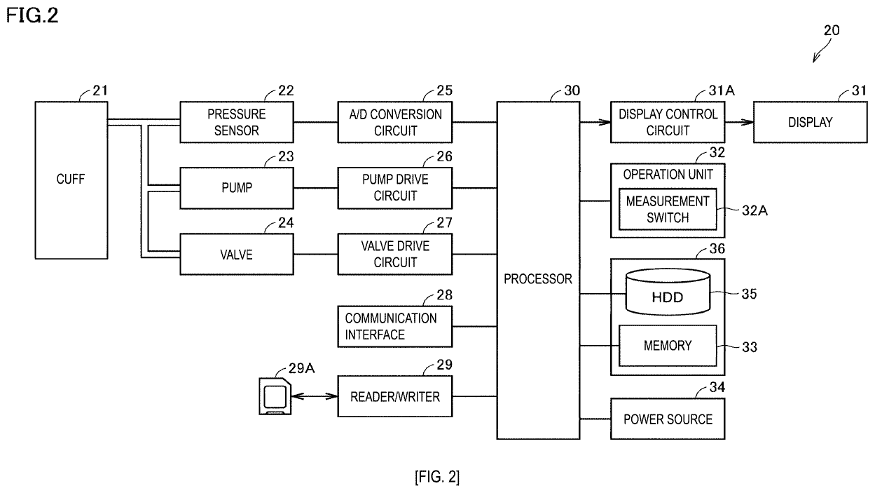

Based on the pulse wave signal superimposed on the cuff pressure signal indicating the cuff pressure detected by a pressure sensor 22, the blood pressure measurement unit 220 adjusts the cuff pressure using the components illustrated in FIG. 2, which will be described later, in accordance with an instruction indicated by a user operation performed on the blood pressure measurement unit that measures the user's blood pressure and the blood pressure monitor. Specifically, the blood pressure measurement unit 220 drives a pump 23 via a pump drive circuit 26 and controls the valve 24 via a valve drive circuit 27. The valve 24 is controlled to open and close to adjust the cuff pressure by discharging or sealing air in the fluid bag.

The blood pressure measurement unit 220 receives the cuff pressure signal detected by the pressure sensor 22 and takes out a pulse wave signal representing the pulse wave of the site to be measured superimposed on the cuff pressure signal. That is, the blood pressure measurement unit 220 detects a time series pulse wave, which is a pressure component superimposed on the cuff pressure signal in synchronization with the pulsation of the heart of the user, from the cuff pressure signal. The analog pulse wave signal measured by the pressure sensor 22 is converted into digital pulse wave information. The interval calculation unit 221 included in the blood pressure measurement unit 220 calculates an interval between adjacent pulse waves in the time series pulse waves constituting the pulse wave information. The interval calculation unit 221 generates interval information 362 indicating an interval between adjacent pulse waves constituting a set in association with each of the sets constituted by two adjacent pulse waves in the time series pulse waves. The interval information 362 is stored in the storage unit 36 by the storing control unit 250. In the present embodiment, the interval between the pulse waves indicates, for example, a period of time from a time when a peak at which the amplitude of the pulse wave signal becomes maximum is detected to a time when a peak of the amplitude of the next pulse wave signal is detected.

The interval calculation unit 221 is an example of an “information acquisition unit” that acquires a feature amount of a pulse wave. Although the interval calculation unit 221 is illustrated as a function included in the blood pressure measurement unit 220 in FIG. 3, it may be provided as a function independent of the blood pressure measurement unit 220. Note that the feature amount is the interval of the pulse wave, but is not limited to the interval.

The blood pressure measurement unit 220 calculates the blood pressure measurement information 363 indicating the user's blood pressure based on the pulse wave signal. The blood pressure measurement information 363 is stored in the storage unit 36, and is output to the output control unit 240 as a measurement result. Specifically, in the blood pressure measurement, the cuff pressure is increased to a predetermined pressure. Based on the pulse wave signal detected in the subsequent depressurization process, the blood pressure measurement unit 220 measures the blood pressure of the user according to the oscillometric method, outputs the measurement result to the output control unit 240, and stores the blood pressure measurement information 363 including the measurement result in the storage unit 36 via the storing control unit 250. Typically, the blood pressure measurement information 363 calculated by the blood pressure measurement unit 220 includes a systolic blood pressure, a diastolic blood pressure, and a pulse rate.

In the AF determination process performed by the AF determination unit 230, a pulse number determination unit 232 analyzes the interval information 362 stored in the storage unit 36 and determines whether or not the number of pulse waves corresponding to the interval information 362 has reached a predetermined number based on the analysis result. In response to the pulse number determination unit 232 determining that the number of pulse waves corresponding to the interval information 362 has reached the predetermined number, the AF determination unit 230 performs a predetermined process based on the interval information 362 corresponding to the predetermined number of pulse waves in the storage unit 36, thereby determining the presence or absence of AF in the pulse waves. The predetermined process is a process for detecting AF, and for example, the presence or absence of AF is determined using a pattern of pulse wave intervals. In the pulse wave signal, the peak (maximum point) of the amplitude for each beat is detected, and the time interval between the peaks of the current beat and the immediately-previous beat is calculated as the pulse wave interval. The interval used for the determination is not limited to the interval between peaks, and may be an interval between rising points of adjacent pulse waves.

A determination result by the above-described AF determination process is output to the output control unit 240, and AF determination information 364 indicating the determination result is stored in the storage unit 36.

Based on the measurement result from the blood pressure measurement unit 220 and the determination result from the AF determination unit 230, the output control unit 240 generates a display data for displaying the blood pressure value and the result of the AF determination, and outputs the display data to a display control circuit 31A. The display control circuit 31A drives the display 31 based on such display data, thereby causing the display 31 to display the blood pressure value and the information of the AF determination.

In addition, in the AF determination process, the selection unit 231 included in the AF determination unit 230 selects a set of pulse waves for which AF determination is performed with high accuracy. Specifically, for each of the plurality of sets of pulse waves indicated by the interval information 362 stored in the storage unit 36, the selection unit 231 deletes, from the interval information 362, a set of pulse numbers corresponding to blood pressure measurement in which the elapsed time from the time when the pulse wave is measured (blood pressure measurement date and time) is equal to or greater than a threshold value.

In the AF determination process, the selection unit 231 compares the peak value of the amplitude of the pulse wave signal with the threshold value for each of the plurality of sets of pulse waves indicated by the interval information 362 stored in the storage unit 36. Based on the comparison result, the selection unit 231 detects a set of pulse waves having an amplitude corresponding to a peak value less than the threshold value, and deletes the detected set from the interval information 362.

The processes of the pulse number determination unit 232 and the AF determination unit 230 are performed on the interval information 362 after the selective deletion of the set of pulse waves by the selection unit 231. As a result, of the pulse wave of the old past measurement date and time or the information of the pulse wave whose amplitude is not sufficient is excluded from the information of the pulse wave used for the determination of AF. Therefore, these pieces of information can be prevented from acting as noise, and the accuracy of AF determination can be improved. As a method of selecting a pulse wave to be deleted, one or both of the selection based on the elapsed time from the measurement time and the selection based on the amplitude of the pulse wave signal may be performed.

According to the blood pressure monitor 20 described above, the determination of AF is performed based on the interval information 362 corresponding to the predetermined number of pulse waves stored in the storage unit 36 when the total number of pulse waves acquired for an individual blood pressure measurement reaches the predetermined number. In this manner, the fact that the total number of pulse waves acquired by measurement reaches a predetermined number, not the number of times of blood pressure measurement, becomes a trigger for an AF determination. Therefore, unlike Patent Literature 1, it is not necessary to perform blood pressure measurement continuously three times for each time of blood pressure measurement for an AF determination, and thus it is possible to reduce the burden on the user.

### Network Configuration

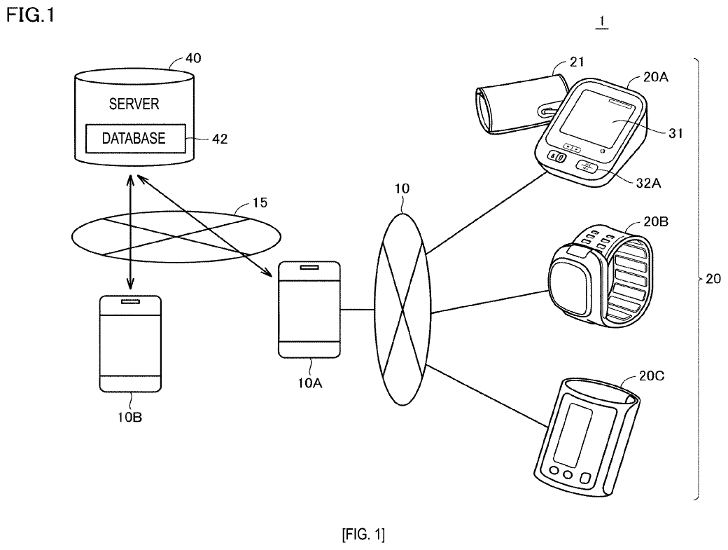

FIG. 1 is a diagram illustrating an example of a network configuration to which the blood pressure monitor 20 according to the present embodiment is applied. Referring to FIG. 1, a network system 1 includes the blood pressure monitor 20, a server 40, and terminals 10A and 10B of portable information processors such as smartphones, which are connectable to a network. The blood pressure monitor 20 communicates with the terminal 10A via the network 10. The terminals 10A and 10B communicate with the server 40 via the network 15. The information measured in the blood pressure monitor 20 may be transferred to the server 40 and stored in the database 42. The network 10, 15 includes various communication networks such as Wi-Fi (registered trade name), a mobile communication network, and the Internet.

The blood pressure monitor 20 may include different types of blood pressure monitors 20A, 20B and 20C. The blood pressure monitor 20 is a stationary upper arm blood pressure monitor in which a main body and a cuff 21 are separate bodies. The blood pressure monitor 20B is a wristwatch-type blood pressure monitor in which the main body and the cuff are integrated. The blood pressure monitor 20C is configured by integrating a cuff and a main body, and is mounted on, for example, an upper arm.

Hereinafter, for convenience of description, the blood pressure monitors 20A, 20B, and 20C may be collectively referred to as the “blood pressure monitor 20”. Any type of blood pressure monitor is configured to be able to perform the above-described blood pressure measurement and AF determination. In the present embodiment, the blood pressure monitor 20A will be described as an example of the blood pressure monitor 20.

The terminals 10A and 10B are, for example, smartphones having a touch panel that constitutes a display. The terminal 10A receives measurement information from the blood pressure monitor 20, displays the received measurement information on the display, and transfers the received measurement information to the server 40 for storage in the database 42. The terminal 10B communicate with the server 40 to receive the measurement information retrieved from the database 42 and display it on a display. Even if the blood pressure monitor 20 is a stationary type, the user can acquire information measured by the blood pressure monitor 20 by using the terminals 10A and 10B carried by the user.

In the present embodiment, the functions of FIG. 3 are implemented in the blood pressure monitor 20, but the implementation method is not limited thereto. For example, the functions may be configured by a plurality of devices such as the blood pressure monitor 20, the server 40, and the terminals 10A and 10B cooperating with each other. When such a plurality of devices cooperate with each other, processing for realizing the function of the blood pressure measurement system can be realized by distributed processing among such a plurality of devices. As distributed processing, for example, among the functions of the blood pressure monitor 20 in FIG. 3, the AF determination unit 230 may be implemented in the terminal 10A or the server 40. Further, the storage unit 36 may be configured in the database 42 of the server 40 or the terminals 10A and 10B. The distribution method is not limited thereto.

### Hardware Configuration

FIG. 2 is a block diagram illustrating an example of a hardware configuration of the blood pressure monitor 20 according to the present embodiment. Referring to FIG. 2, the blood pressure monitor 20 includes the main body and a cuff 21 as main components. The cuff 21 contains a fluid bag of air. The main body includes a processor 30, an air system component constituting a “cuff pressure adjusting unit” for blood pressure measurement, an A/D conversion circuit 25, the pump drive circuit 26, the valve drive circuit 27, the display 31 to which a display control circuit 31A is connected, the storage unit 36, an operation unit 32 for receiving user operations on the blood pressure monitor 20, a communication interface 28 for communicably connecting the blood pressure monitor 20 to a network, a reader/writer 29 to which a nonvolatile storage media such as a memory card 29A is removably attached, and a power source 34.

The processor 30 constitutes an arithmetic processing circuit such as a CPU (Central Processing Unit) or an MPU (Micro Processing Unit). The processor 30 realizes the processing of the blood pressure monitor 20 by reading and executing a program from the storage unit 36. For example, the processor 30 controls the driving of the pump 23 and the valve 24 in response to an operation signal from the operation unit 32. In addition, the processor 30 calculates a blood pressure value using an algorithm for blood pressure calculation by an oscillometric method and displays the blood pressure value on the display 31.

The storage unit 36 includes a nonvolatile storage media such as HDD 35 and the memory 33. The memory 33 includes a volatile or nonvolatile storage medium. The memory 33 includes, for example, a RAM (Random Access Memory), a ROM (Read-Only Memory), a flash memory, and the like. The storage unit 36 stores a program for controlling the blood pressure monitor 20, data used to control the blood pressure monitor 20, setting data for setting various functions of the blood pressure monitor 20, and information on the measurement results of blood pressure values. The memory 33 is also used as a working memory or the like for the processor 30 to expand and execute the program read from the storage unit 36.

The air system component increases or decreases the cuff pressure indicating the internal pressure of the cuff 21 attached to the measurement site of the user. Specifically, the air system components include the pressure sensor 22 for detecting the cuff pressure, which is the pressure inside the fluid bag, so that air can be supplied to or discharged from the fluid bag contained in the cuff 21 through air piping, and the pump 23 and the valve 24 as an expansion/contraction mechanism for expanding and contracting the fluid bag.

The pressure sensor 22 detects the pressure (cuff pressure) in the fluid bag and outputs a signal (cuff pressure signal) corresponding to the detected pressure to the A/D conversion circuit 25. The pressure sensor 22 is, for example, a piezo-resistance pressure sensor, and is fluidly connected to the pump 23, the valve 24, and the fluid bag contained in the cuff 21 via air piping. The pump 23 supplies air as a fluid to the fluid bag through an air pipe in order to increase the cuff pressure. The valve 24 opens and closes to control the cuff pressure by discharging air from the fluid bag through the air piping or sealing air in the fluid bag.

The A/D conversion circuit 25 converts the output value (e.g., electrical resistance) of the pressure sensor 22 from an analog signal to a digital signal and outputs the signal to the processor 30. In this example, the processor 30 functions as an oscillation circuit that oscillates at a frequency corresponding to a change in electrical resistance due to the piezoresistive effect from the pressure sensor 22, and acquires a signal representing the cuff pressure in accordance with the oscillation frequency. The pump drive circuit 26 controls the drive of the pump 23 based on a control signal given from the processor 30. The valve drive circuit 27 controls the opening and closing of the valve 24 based on a control signal given from the processor 30.

When blood pressure is measured according to the oscillometric method, the following operation is generally performed. Specifically, the cuff 21 is wrapped around the site to be measured (wrist, arm, etc.) of the user (subject) in advance, and the pump 23 and valve 24 are controlled to pressurize the cuff 21 during measurement. In this pressurization process, when the cuff pressure is increased to the predetermined pressure Cp, the pump 23 is stopped and the valve 24 is controlled to be gradually opened. As a result, the cuff pressure is reduced. In this depressurization process, the change in arterial volume occurring in the artery at the measurement site is extracted as a pulse wave signal superimposed on the cuff pressure signal. The systolic blood pressure and the diastolic blood pressure are calculated based on the change in amplitude of the pulse wave signal (mainly the rise and fall) accompanying the change in cuff pressure during the depressurization process. Such blood pressure measurement is not limited to the case of being performed in the depressurization process, and may be performed using a pulse wave signal superimposed on a cuff pressure signal detected in the pressurization process.

The operation unit 32 receives an operation performed on the blood pressure monitor 20 by the user and outputs an operation signal indicating the received user operation to the processor 30. The processor 30 outputs a command based on the operation signal to each unit. The operation unit 32 includes a measurement switch 32A that is operated to instruct the start of blood pressure measurement. The operation unit 32 may include other types of switches or buttons.

When the measurement switch 32A is operated, the processor 30 controls the air system components so that the measurement site is compressed by the cuff 21, and calculates the blood pressure value according to the oscillometric method. If the measurement switch 32A is operated again while such a blood pressure measurement is being performed, the processor 30 stops the blood pressure measurement.

The reader/writer 29 reads a program or data from a memory card 29A attached thereto. The processor 30 stores the read program or data in the storage unit 36. Further, the reader/writer 29 writes the information such as the measurement result read from the storage unit 36 by the processor 30 into the attached memory card 29A.

The display 31 displays various information including blood pressure measurement results, AF determination information, etc., based on display data from the display control circuit 31A. The communication interface 28 includes, for example, a network interface card (NIC), and controls exchange of information between the blood pressure monitor 20 and other devices (the terminals 10A and 10B and the server 40). The power source 34 supplies power to the processor 30 and each piece of hardware.

### Flowchart and Display Example

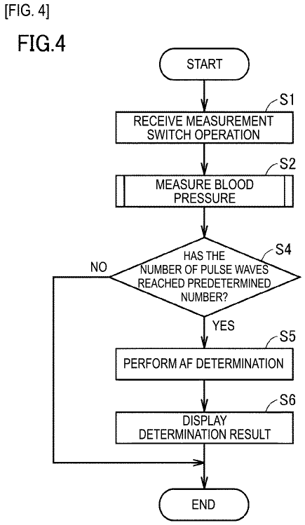

FIG. 4 is a flowchart illustrating an example of processing related to measurement by the blood pressure monitor 20 according to the present embodiment. At the start of this process, the cuff 21 of the blood pressure monitor 20 is attached (wrapped) to the measurement site of the user.

Referring to FIG. 4, the processor 30 of the blood pressure monitor 20 receives an operation signal based on a user operation of the measurement switch 32A from the operation unit 32 (step S1). The processor 30 starts the blood pressure measurement process (step S2) in response to the operation signal. In the blood pressure measurement process, the interval information 362 is stored in the storage unit 36. The blood pressure measurement process will be described in detail later.

The processor 30 detects the number of pulse waves corresponding to the interval information 362 stored in the storage unit 36, and determines whether or not the detected number of pulse waves reaches a predetermined number (step S4). When the processor 30 determines that the number of pulse waves has reached the predetermined number (YES in step S4), the processor 30 performs the above-described AF determination process (step S5). In the AF determination process of step S5, the processor 30 extracts interval information corresponding to the predetermined number of pulse waves from the interval information 362 of the storage unit 36, and determines the presence or absence of AF in the pulse waves based on the extracted interval information.

The processor 30 outputs display data for displaying the result of the AF determination in step S5 on the display 31 to the display control circuit 31A (step S6).

On the other hand, when the processor 30 does not determine that the number of pulse waves has reached the predetermined number (NO in step S4), the AF determination in step S5 and the process of displaying the determination result (steps S5 and S6) are not performed, and the process ends.

FIG. 5 is a flowchart illustrating an example of the blood pressure measurement processing of FIG. 4. In FIG. 5, the processor 30 initializes the pressure sensor 22 with the pump 23 off (stopped) and the valve 24 open (step S21). When initializing the pressure sensor 22, the current output value of the pressure sensor 22 is set as a value equivalent to atmospheric pressure. In step S21, the processor 30 initializes the working memory area 361 of the storage unit 36.

The processor 30 closes the valve 24 via the valve drive circuit 27 (step S22). The processor 30 then turns on (starts) the pump 23 via the pump drive circuit 26, and starts pressurizing the cuff 21 (fluid bag) at a predetermined pressurization speed (step S23).

The processor 30 compares the cuff pressure indicated by the cuff pressure signal detected by the pressure sensor 22 with the predetermined pressure Cp, and determines whether or not the cuff pressure has reached the predetermined pressure Cp based on the result of the comparison (step S24). If it is not determined that the cuff pressure has reached the predetermined pressure Cp (NO in step S24), the process returns to step S23 and the cuff 21 is pressurized at a predetermined pressurization rate.

If it is determined that the cuff pressure has reached the predetermined pressure Cp (YES in step S24), the processor 30 turns off (stops) the pump 23 via the pump drive circuit 26 (step S25). Thereafter, the processor 30 gradually opens the valve 24 via the valve drive circuit 27 so that the cuff pressure of the cuff 21 indicated by the cuff pressure signal detected by the pressure sensor 22 is reduced at a predetermined pressure reducing rate (step S26).

During the depressurization process in which depressurization is performed at this predetermined rate, the processor 30 extracts a pulse wave signal from the cuff pressure signal detected by the pressure sensor 22, and attempts to calculate the maximum blood pressure (systolic blood pressure) and minimum blood pressure (diastolic blood pressure) based on the extracted pulse wave signal, and determines whether the blood pressure calculation is complete (step S28). The processor 30 stores the pulse wave signal detected in the depressurization process in the working memory area 361. When the processor 30 determines based on the pulse wave signals stored in the working memory area 361 that the blood pressure calculation cannot be completed due to insufficient pulse wave signals being obtained (NO in step S28), the processor 30 returns to step S26. When it is determined that the blood pressure calculation is completed (YES in step S28), the processor 30 sets the valve 24 to be fully opened via the valve drive circuit 27 so that the air in the cuff 21 is rapidly discharged (step S29). The processor 30 displays the blood pressure value (measurement result) measured in step S27 on the display 31 (step S30). The blood pressure measurement information 363 indicating the blood pressure value (measurement result) is stored in the storage unit 36.

The processor 30 calculates the interval between the pulse waves for each set of the plurality of pulse waves indicated by the pulse wave signals stored in the working memory area 361 (step S31), and stores the interval information 362 indicating the calculated interval between each set in the storage unit 36 (step S32).

As described above, in the present embodiment, for each blood pressure measurement, the interval information 362 is acquired for a plurality of pulse waves corresponding to the pulse wave signal used for calculation of the blood pressure. Thus, the interval information 362 acquired for an individual blood pressure measurement is stored in the storage unit 36 in association with the measurement date and time of the corresponding blood pressure measurement.

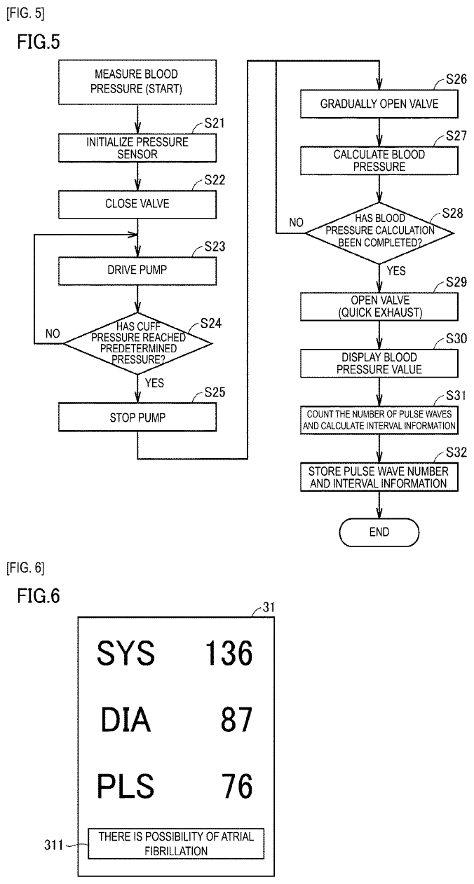

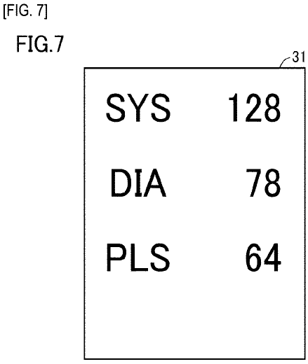

FIGS. 6 and 7 are diagrams illustrating display examples of information on the display 31 according to the present embodiment. FIG. 6 illustrates a display example when it is determined that AF is present in a case where the AF determination process (step S5) is performed after the blood pressure measurement (step S2). In FIG. 6, the blood pressure measurement results (systolic blood pressure SYS, diastolic blood pressure DIA, and pulse rate PLS) and a message 311 indicating that it is determined that AF is present are displayed.

On the other hand, FIG. 7 illustrates a display example of a case where the AF determination process (step S2) is not performed after the blood pressure measurement (step S5) or a case where the AF determination process (step S2) is performed after the blood pressure measurement (step S5) and it is determined that “AF is not present”. In FIG. 7, only the blood pressure measurement results (systolic blood pressure SYS, diastolic blood pressure DIA, and pulse rate PLS) are displayed, and the AF determination result is not displayed. In a case where the AF determination process (step S5) is performed after the blood pressure measurement (step S2) and it is determined that “AF is not present”, a message “AF is not present” may be displayed on the screen of FIG. 7.

### Example of AF Determination

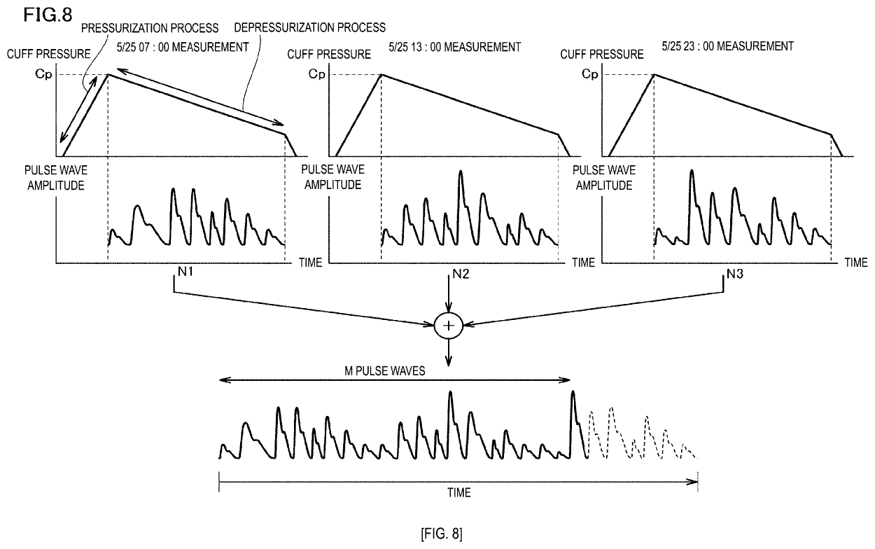

FIG. 8 is a diagram illustrating an example of a plurality of pulse waves used for an AF determination according to the present embodiment. In the present embodiment, the interval information 362 of the pulse wave acquired in each of the plurality of blood pressure measurements is integrated, and the determination of AF is performed based on the integrated interval information 362.

In FIG. 8, when the blood pressure measurement is performed three times in the morning (7:00), the daytime (13:00), and the evening (23:00) within one day, the pulse wave interval information 362 is acquired in each blood pressure measurement and stored in the storage unit 36. In this case, in each of the morning and daytime blood pressure measurements, the interval information 362 corresponding to each blood pressure measurement is stored in the storage unit 36. When the blood pressure measurement is performed in the daytime, in step S4 of FIG. 4, the pulse number determination unit 232 determines that the number of pulse waves corresponding to the interval information 362 stored in the storage unit 36 does not reach M necessary for the AF determination (NO in step S4 of FIG. 4), and the AF determination process is not performed. In the subsequent evening blood pressure measurement, the interval information 362 corresponding to the blood pressure measurement is stored in the storage unit 36. When the blood pressure measurement is performed in the evening, in step S4 of FIG. 4, the pulse number determination unit 232 determines that the number of pulse waves corresponding to the interval information 362 stored in the storage unit 36 has reached M necessary for the AF determination (YES in step S4 of FIG. 4). That is, the pulse number determination unit 232 counts the number of pulse waves corresponding to the interval information 362 in the storage unit 36, that is, the total number of pulse waves obtained by integrating the pulse waves acquired by the morning, daytime, and evening blood pressure measurements as (total number=N1+N2+N3), and determines that the condition of (total number≥M) is satisfied (YES in step S4 in FIG. 4). As described above, in FIG. 8, when the blood pressure measurement is performed in the evening (step S2 in FIG. 4), the AF determination unit 230 integrates the interval information 362 corresponding to the pulse waves (M pulse waves) acquired by the blood pressure measurement in the morning, daytime, and evening stored in the storage unit 36, and performs the AF determination (step S5 in FIG. 4) based on the integrated information. The M pulse waves described above include a plurality of pulse waves acquired in one or a plurality of blood pressure measurements (step S2 in FIG. 4). To be more specific, the M pulse waves include a plurality of pulse waves acquired in one or a plurality of blood pressure measurements (step S2 in FIG. 4) performed after the previous AF determination process is performed.

### Advantages of Embodiment

The AF is a disorder that causes severe cardiac disease and requires early initiation of treatment. However, since many are asymptomatic, the onset of treatment is delayed without being aware of AF. Therefore, it is desirable to routinely screen for the presence or absence of AF. Such screening is performed on the basis of electrocardiogramedical institution or the like, and the opportunity for discovering AF is limited. On the other hand, since the home blood pressure monitor 20 according to the present embodiment can screen (determine) AF using the pulse wave acquired by the blood pressure measurement by using the opportunity of the user to measure the blood pressure, it is possible to provide many opportunities for discovering AF. Since some patients with AF do not always have symptoms of AF but have symptoms due to environmental factors such as alcohol, stress, and lack of sleep, the mechanism for providing many opportunities for AF discovery as in the present embodiment can also provide support information for effectively determining the start of treatment for such cases.

The blood pressure monitor 20 according to the present embodiment is configured to perform the AF determination process when it is determined that the total number of pulse waves corresponding to the interval information 362 has reached the predetermined number. This configuration eliminates the need to continuously measure three times each time the blood pressure is measured as in Patent Literature 1. As a result, according to the present embodiment, the user is not burdened with a longer time required for measurement, repeated compression of the measurement site at the predetermined pressure Cp equal to or higher than the systolic blood pressure, and the like.

In the present embodiment, unlike Patent Literature 1, even if three consecutive blood pressure measurements are not required, the selection unit 231 excludes information about old past measurement dates and times or pulse waves with insufficient amplitudes from the interval information 362 used for an AF determination. This prevents these pieces of information from affecting the determination as noise, improves the determination accuracy.

In addition, since the present embodiment is configured to store not the pulse wave information itself but only the pulse wave interval information 362 in the storage unit 36 for the AF determination, it is possible to save a memory capacity for storing information necessary for the determination.

### Other Embodiments

(1) In the above-described embodiment, a program is provided that causes a computer, such as the processor 30 of the blood pressure monitor 20, to execute the processes described in the above flowcharts. Such a program can be provided as a program product by being recorded on a non-transitory computer-readable recording medium such as a flexible disk, a CD-ROM (Compact Disk Read Only Memory), a secondary storage device, a main storage device, or a memory card 29A that comes with the computer. Alternatively, such a program can be provided by being recorded on a recording medium such as the HDD 35 built into the computer. Moreover, the program can be provided to such a computer by downloading it from a distribution server (not shown) via the network 10, 15.
(2) The configuration exemplified as the embodiment described above is an example of a configuration of the present invention, and the configuration can be combined with other known technology, and may be partially omitted or modified within the scope not deviating from the gist of the present invention. Furthermore, the processes and configurations described in other embodiments may be employed as appropriate in the embodiments described above.

### [Supplementary Notes]

As described above, the present embodiments include the following disclosures.

### Configuration 1

A blood pressure monitor (20) including:
a cuff pressure adjustment unit (23,24) configured to increase or decrease a cuff pressure indicating an internal pressure of a cuff attached to a measurement site of a user;
a cuff pressure detector (22) configured to detect a cuff pressure signal indicating the cuff pressure;
a blood pressure measurement unit (220) configured to measure a blood pressure of the user based on a pulse wave signal superimposed on a cuff pressure signal detected in a process of increasing or decreasing the cuff pressure;
an information acquisition unit (221) configured to acquire, for an individual blood pressure measurement, pulse wave feature information representing a feature amount of a pulse wave from the pulse wave signal superimposed on the cuff pressure signal detected in the blood pressure measurement;
a storing unit (36) configured to store pulse wave feature information acquired for the individual blood pressure measurement;
a pulse number determination unit (232) configured to determine the number of pulse waves having a feature amount represented by pulse wave feature information for the individual blood pressure measurement; and
an atrial fibrillation determination unit (230) configured to determine atrial fibrillation in the pulse wave based on the pulse wave feature information in the storing unit when the total number of the pulse waves for the individual blood pressure measurement reaches a predetermined number.

### Configuration 2

The blood pressure monitor (20) according to configuration 1, wherein the pulse number determination unit (232) is configured to
determine the number of pulse waves for the individual blood pressure measurement by excluding, from the pulse wave feature information for the individual blood pressure measurement, pulse wave feature information corresponding to a blood pressure measurement in which an elapsed time from when the blood pressure measurement is performed is equal to or greater than a threshold value.

### Configuration 3

The blood pressure monitor (20) according to configuration 1 or 2, wherein the feature amount of the pulse wave represented by the pulse wave feature information includes an interval between pulse waves represented by the pulse wave signal superimposed on the cuff pressure signal detected in the blood pressure measurement.

### Configuration 4

The blood pressure monitor (20) according to configuration 3, wherein the interval between pulse waves includes a time interval between maximum values of amplitudes in adjacent pulse waves.

### Configuration 5

The blood pressure monitor (20) according to any one of configurations 1 to 4, wherein
the pulse number determination unit is configured to determine the number of pulse waves for the individual blood pressure measurement by excluding pulse wave feature information corresponding to a pulse wave in which an amplitude of a pulse wave having a feature amount represented by the pulse wave feature information for the individual blood pressure measurement is equal to or less than a threshold value.

### Configuration 6

The blood pressure monitor (20) according to any one of configurations 1 to 5, wherein
the pulse number determination unit is further configured to
determine whether or not the total number of pulse waves corresponding to the pulse wave feature information for the individual blood pressure measurement in the storing unit reaches the predetermined number.

The embodiments disclosed herein are illustrative in all respects and are not intended as limitations. The scope of the present invention is indicated not by the descriptions above but by the claims and includes all meaning equivalent to the scope and changes within the scope.

### REFERENCE NUMERALS LIST

1 Network system
10, 15 Network
10A, 10B Terminal
20 20A, 20B, 20C Blood pressure monitor
21 Cuff
22 Pressure sensor
23 Pump
24 Valve
25 A/D conversion circuit
26 Pump drive circuit
27 Valve drive circuit
28 Communication interface
29 Reader/writer
29A Memory card
30 Processor
31 Display
31A Display control circuit
32 Operation unit
32A Measurement switch
33 Memory
34 Power source
35 HDD
36 Storage unit
40 Server
42 Database
220 Blood pressure measurement unit
221 Interval calculation unit
230 AF determination unit
231 Selection unit
232 Pulse number determination unit
240 Output control unit
250 Storing control unit
311 Message
361 Working memory area
362 Interval information
363 Blood pressure measurement information
364 Determination information
Cp Predetermined pressure

What is claimed is:

1. A blood pressure monitor comprising:
a cuff pressure adjustment unit configured to increase or decrease a cuff pressure indicating an internal pressure of a cuff attached to a measurement site of a user;
a cuff pressure detector configured to detect a cuff pressure signal indicating the cuff pressure;
a blood pressure measurement unit configured to measure a blood pressure of the user based on a pulse wave signal superimposed on a cuff pressure signal detected in a process of increasing or decreasing the cuff pressure;
an information acquisition unit configured to acquire, for an individual blood pressure measurement, pulse wave feature information representing a feature amount of a pulse wave from the pulse wave signal superimposed on the cuff pressure signal detected in the blood pressure measurement;
a storing unit configured to store pulse wave feature information acquired for the individual blood pressure measurement;
a pulse number determination unit configured to determine the number of pulse waves having a feature amount represented by pulse wave feature information for the individual blood pressure measurement;
a total number determination unit configured to determine whether a total number of pulse waves obtained by integrating pulse waves for individual blood pressure measurements has reached a predetermined number; and
an atrial fibrillation determination unit configured to determine atrial fibrillation in a pulse wave based on the pulse wave feature information corresponding to the integrated pulse waves in the storing unit every time the total number is determined to have reached a predetermined number, wherein
the total number determination unit is configured to determine whether the total number of pulse waves obtained by integrating pulse waves and determined by the pulse number determination unit for one or more blood pressure measurements after a previous determination of atrial fibrillation has reached the predetermined number.

2. The blood pressure monitor according to claim 1, wherein the pulse number determination unit is configured to determine the number of pulse waves for the individual blood pressure measurement by excluding, from the pulse wave feature information for the individual blood pressure measurement, pulse wave feature information corresponding to a blood pressure measurement in which an elapsed time from when the blood pressure measurement is performed is equal to or greater than a threshold value.

3. The blood pressure monitor according to claim 1, wherein the feature amount of the pulse wave represented by the pulse wave feature information includes an interval between pulse waves represented by the pulse wave signal superimposed on the cuff pressure signal detected in the blood pressure measurement.

4. The blood pressure monitor according to claim 3, wherein the interval between pulse waves includes a time interval between maximum values of amplitudes in adjacent pulse waves.

5. The blood pressure monitor according to claim 4, wherein the pulse number determination unit is configured to determine the number of pulse waves for the individual blood pressure measurement by excluding pulse wave feature information corresponding to a pulse wave in which an amplitude of a pulse wave having a feature amount represented by the pulse wave feature information for the individual blood pressure measurement is equal to or less than a threshold value.
